(19) United States
(12) Patent Application Publication
(10) Pub. No.: US 2021/0202124 A1
(43) Pub. Date: Jul. 1, 2021

(54) Spherical structures for imaging, ablation, antennas, detectors,optical tweezing, and quantum operations

(71) Applicant: Asaf Farhi, Orlando, FL (US)

(72) Inventor: Asaf Farhi, Orlando, FL (US)

(21) Appl. No.: 16/730,965

(22) Filed: Dec. 30, 2019

(51) Int. Cl. G21K 1/00; H01Q 1/36; G02B 21/00; A61B 18/18; G01N 33/50

(52) U.S. Cl. CPC G21K 1/006; H01Q 1/36; G02B 21/006; H01Q 9/16; A61B 18/18; G01N 33/5005; G02B 21/0072

(57) ABSTRACT

A spherical layer apparatus for imaging, ablation, optical tweezing, and quantum operations is described. In addition, a spherical device that can scatter and absorb electromagnetic radiation effectively, enhance emission and absorption of nearby molecules and atoms, and direct radiation toward a radiation source. The apparatus and device can work in the same setup.

### TECHNICAL FIELD

The patent is the field of optics and can be applicable to additional wave-physics fields such as acoustics, matter waves etc. We describe a spherical shell apparatus to perform three-dimensional imaging and ablation and a spherical device that can absorb and scatter radiation very efficiently, enhance interactions of nearby atoms and molecules, and direct radiation towards a radiation source. Potential applications of the spherical device are ablating human cells (e.g., cancer cells) or human-cell components (e.g., microtubules) and omni-directional antenna and detector.

### BACKGROUND ART

The focal spot size that can be achieved by uniformly illuminating a circular aperture in the scalar approximation is given by an Airy disc, which is the Fourier Transform of a circular window [1]. The full width at half maximum (FWHM) of this function is 1.02λ/NA, where λ is the wavelength and NA is the numerical aperture (NA≲1). This size is associated with the lateral axes and in the axial axis the FWHM is 2.5-3 times larger due to the fact that a smaller range of k_{z}s is involved. For gaussian beams, however, the focal spot is larger and depends on the width of the beam. The optimal lens resolution enables to image most biological cells but not viruses, proteins, and smaller molecules. Techniques such as confocal microscopy, structured illumination, beam shaping, and hyperlens imaging have been used to increase the lateral resolution [2 4]. In a 4π microscope the sample is illuminated from both sides and better resolution in the axial axis can be achieved [5]. However, in this setup side lobes are generated and the optical system needs to be realigned before every measurement in order for the focal spots to merge. Techniques based on fluorescence such as STED [6], and PALM and STORM [7, 8] enable subwavelength resolution by stimulating emission at another frequency using an additional torus-like illumination and by activating subsets of fluorescent molecules, which enables to accurately calculate the molecule locations, respectively. Maxwell fisheye is a spherical lens with a radius-dependent refraction index in which all light rays emitted from a point meet at the antipodal point. The possibility of obtaining subwavelength resolution inside this setup has been the subject of recent works [9, 10]. Time reversal of waves has also been applied for generating a subwavelength focal spot [11, 12]. Finally, methods based on evanescent waves to enhance resolution such as near-field imaging and negative-refractive index lens enable subwavelength focusing usually for two-dimensional imaging [13]. Here, we utilize a resonant spherical layer to localize far-field light in several settings. We first situate the spherical layer in a uniform medium and excite it with a point current. This setup generates a three-dimensional free-space subwavelength focal that has very minor side lobes. Since it is composed of one “lens” it may not need to be aligned. In excitation-collection mode the effective focal spot is further minimized and there are almost no side lobes. We then suggest two directions to localize far-field with deep-subwavelength resolution using a setup of a spherical layer.

Time reversal of waves has been utilized for various interesting applications such as wave localization [11, 12, 14, 15] and coherent-perfect absorption [16]. Recently, it was shown that the time reversal of a source, in the presence of a near-perfect absorber, results in a subwavelength focal spot [12]. In order to generate the time reversal of a wave generated by a source, one would have to let the wave propagate from the source, “freeze” time, and generate discrete sources on a spherical envelope modulated according to the wave amplitude. Here, we will utilize the resonant-spherical layer setup to generate the spatially-continuous time reversal wave of sources, enabling its use in electrodynamics.

Degeneracies of eigenvalues can arise from a symmetry of the system or from a special feature of the system. While the first type of degeneracy is widely known (e.g., m degeneracy in spherical multipoles), the second, called accidental degeneracy, is more exotic and includes phenomena such as Landau levels [17], exceptional points [18-22] and the accumulation point of the eigenpermittivities of evanescent modes [23]. Degeneracies are associated with a strong response of the system as several modes are excited. In exceptional points for example, the degeneracy is usually second or third order and can lead to enhancement of emission from a molecule by two orders of magnitude due to enhancement of the density of states [24]. In addition, the accumulation point of the eigenpermittivities of the evanescent modes can enhance the field (and emission) for a source that is very close to a metal-dielectric interface. Here we will show analytically and numerically that a spherical structure with a radius larger than 20λ exhibits infinite asymptotic all-even and all-odd TE/TM degeneracies of the second type. These degeneracies are associated with far field and dielectric spherical structures, in some cases with gain.

In a homogeneous medium the continuous-wave source-free solutions of Maxwell's equation are plane waves, vector spherical harmonics, and vector cylindrical harmonics. It was recently shown that similarly to the situation in phased arrays in which plane currents proportional to a homogeneous medium source-free solution with a planar geometry generate the same function, currents proportional to a vector spherical harmonic (VSH) on a spherical surface generate the same VSH. Interestingly, a TM l=1 VSH near the origin has a subwavelength far-field focal spot [25], which is smaller in volume by a factor of ˜27 compared with the focal spot that can be achieved by uniformly illuminating a lens. For a medium with a refractive index larger than 1, the TM l=1 field will have even a smaller focal spot. Generating this mode by oscillating currents can be thought of as a continuous-wave time reversal of the field of an oscillating dipole at the origin. Importantly, generating these VSH propagating towards the origin are the time reversal of the atomic and molecular multipole transitions. It is thus of interest to generate these modes near the origin. However, the spatial distributions of these VSH are complex and a setup of currents modulated accordingly is infeasible.

### SUMMARY OF THE INVENTION

High-resolution field localization in three dimensions is one of the main challenges in optics and has immense importance in fields such as chemistry, biology, and medicine. Time-reversal symmetry of waves has been a fertile ground for applications such as generating a subwavelength focal spot and coherent-perfect absorption. However, in order to generate the time reversed signal of a monochromatic source discrete sources that are modulated according to the wave amplitude on a spherical envelope are required, rendering it applicable only in acoustics. Here we approach these challenges by introducing a spherical layer with a resonant permittivity, which naturally generates the spatially continuous time-reversed signal of an atomic and molecular multipole transition at the origin. We start by utilizing a spherical layer with a resonant TM l=1 permittivity situated in a uniform medium to generate a free-space-subwavelength focal spot at the origin. We remove the degeneracy of the eigenfunctions of the composite medium by situating a point current source (or polarization) directed parallel to the spherical layer, which generates a focal spot at the origin independently of its location. The free-space focal spot has a full width at half maximum of 0.4λ in the lateral axes and 0.58λ in the axial axis, which is tighter by a factor of \sqrt{2} in each dimension in excitation-collection mode, overcoming the λ/2 far-field resolution limit in three dimensions. This setup can also find applications in optical tweezing since the focal-spot size is optimal. We then suggest two setups to localize electric field with deep-subwavelength resolution in three dimensions using a setup of a spherical layer with the applications of imaging and ablation. In the first we move the system away from resonance and introduce a particle that will bring the system to a resonance when it is located at the origin, enabling to resolve particle locations with high resolution. In the second we introduce an atom or molecule that when situated at the origin will lead to strong light-matter interaction, enabling to resolve their location with very high resolution (we can use a similar approach as in the first). Since the imaginary part of the eigenvalue is also realized in the physical parameter and the setup can he in an exact resonance, it can also open avenues in fields such as cavity QED, entanglement, and quantum information. In addition, we show that spherical structures exhibit a new type of degeneracy in which an infinite number of eigenvalues asymptotically coalesce. This high degeneracy results in a variety of optical phenomena such as strong scattering and enhancement of absorption and emission from an atom or molecule by orders of magnitude compared with a standard resonance. In addition, the radiation from a spherical structure is directed towards the source due to constructive interference of the modes in this direction, with applications of an omnidirectional antenna and detector.

### Technical Problem

The resolution in standard microscopy techniques enables to image most biological cells but not viruses, proteins, and smaller molecules. Thus, most of the information remains hidden, hindering visualization and understanding of interactions and mechanisms in biology, medicine, and chemistry. Similarly, the ablation resolution is limited by the diffraction limit. Targeting cells with electromagnetic radiation can be performed using nanoparticles. However, such nanoparticles are usually metallic and can be unsafe to the human body, are active at optical frequencies that cannot penetrate deeply into the human body, and are based on near-field phenomena, thereby affecting only cells that in their very close proximity. Moreover, metallic particles have losses and are slightly off resonance (quasistatic resonance requires real permittivity). In order to compensate gain is needed, which is challenging experimentally. In addition, if there is electromagnetic interaction in the human body (the interactions that are well understood are up to 1 nm and there are phenomena at a larger distance) it is expected to be at infrared frequencies. Interfering with such interactions may require objects inside the body that can interact strongly at infrared frequencies. Antennas and detectors have irrationality that allows them to transfer and detect energy only at a given direction. Thus, the coverage is only partial and e.g., detection of objects in other directions can be challenging.

### Solution to the Problem

We suggest to utilize a spherical-layer apparatus to perform imaging and ablation with a better resolution. When situating a spherical layer with a resonant permittivity in a uniform medium, the generated focal spot is smaller than the one that can he generated by a lens. In imaging the light can be collected from outside of the spherical layer. Then, we move the system away from resonance by e.g. slightly changing the permittivity of the spherical layer and introduce a spherical particle that when situated at the origin will result in a system resonance. We suggest to utilize this three-body resonance mechanism as a method to localize light with high spatial resolution (when the particle will he at the origin there will he strong intensity of electromagnetic radiation also outside the spherical layer). We then situate an atom or molecule at the origin and suggest that the time reversal of an emission process will result in field that is spatially correlated with the transition current , is deep subwavelength, and optimal for driving the transition. Thus, an interaction of an atom or a molecule with a spherical layer can lead to a strong interaction, large Rahi splitting etc., that can enable to distinguish between atoms/molecules at the origin and slightly shifted with a very high resolution. In addition, we show that spherical structures e-exhibit an infinite-asymptotic degeneracy of their eigenpermittivities that results in strong scattering and absorption, enhancement of emission and absorption of nearby atoms and molecules, and radiation that is directed towards the source. They can be thus used as particles for ablating e.g., cancer cells and cell-components also at infrared frequencies. They can also be used as antennas and detectors in all directions.

### Advantageous Effects of Invention

The focal spot that is generated in the setup of a spherical layer in a host medium is smaller than the one generated by a lens and the setup overcomes the diffraction limit in three dimensions in imaging. In addition, it requires only one source and there is no need to align the system, and there are almost no side lobes in imaging. When introducing a particle in a three-body resonance mechanism the resolution is higher since the intensity increases significantly when the particle is at the origin, enabling to image such particles and ablate their surrounding with high resolution. When introducing an atom or a molecule and utilizing the strong interaction at the origin the resolution can be extremely high. The spherical structures with the infinite degeneracy respond very strongly and can be dielectric. This is a degeneracy that is utilized also for the far field, which is very unique. The spherical structures can enhance emission and absorption of nearby molecules and atoms, scatter and absorb strongly, and function as omnidirectional antennas and detectors (in all directions, unlike the standard definition of omnidirectional in the context of antennas).

### BRIEF DESCRIPTION OF THE DRAWINGS

FIG. 1 is a plot of |E|² of a TM l=1 eigenmode near the origin for a setup of a spherical layer (white) with an oscillating dipole. The external ellipse, is the focal spot of an Airy disc (green). The spherical layer has a permittivity that is close to an eigenpermittivity ∈₁≈∈_{1,TM l-1} and the host-medium permittivity is ∈₂=1.

FIG. 2 is a plot of |E|² of the TM l=1 mode that exists without a source for a setup of a spherical layer in vacuum for r₁=0.7, r₂=1.4 μm, ∈₁=1.5, and ωₗ₋₁/2π=7.92781·10¹⁴+7.397·10⁷i.

FIG. 3 is a plot of |E|² and E (arrows) for a setup of a spherical layer in vacuum with an oscillating dipole source. r₁=0.7, r₂=0.9 μm, ∈₁=1.75-0.7i, r₀=1 \grave{x}μm, p=1 \grave{z}mA and λ=430 nm.

FIG. 4 is a plot of |E|² for a setup of a spherical layer in vacuum with a current loop and ∈=1.45-0.57i, r₁=1.7, r₂=2 μm, λ=430 nm, r₀=2.2 μm.

FIG. 5 is a plot of |E|² for a capped spherical layer with a current loop and r₁=3.4 μm, r₂=4 μm, r₀=4.4 μm, ∈₁=1.82+0.20i., λ=430 nm, J=1A/m²\grave{z}.

FIG. 6 is a plot of |E|⁴ for for a setup of a spherical layer in vacuum with a current loop and ∈₁=1.45-0.57i, r₁=1.7, r₂=2 μm, λ=430 nm, r₀=2.2 μm.

FIG. 7 is a plot of ∈_{1lTM} as a function of the sphere radius for a sphere in vacuum at λ=430 nm.

### DESCRIPTION OF EMBODIMENTS

1. A spherical layer with a permittivity dose to a TM l=1 resonance, possibly a laser source outside of the spherical layer, and a sample inside the spherical layer. The setup may require field at another frequency to generate gain within the spherical layer. For imaging, a device that collects light outside of the spherical layer.
2. Similarly to 1 with a capped spherical layer.
3. In addition to 1 situating spherical particles without gain (that result in a resonance when they are at the origin) in a sample, and imaging them or ablating their surrounding when they are at the origin.
4. In addition to 1tuning the spherical layer to a resonance of a spherical layer—atom or molecule and imaging a sample containing these molecules. When the atom/molecule is at the origin all the light can be absorbed by them and then emitted leading to a strong signal that can enable to resolve them with very high resolution. Also, a spherical layer with a large radius and asymptotic degeneracy or another cavity that will time reverse the transition field and interact strongly with the atom molecule.
5. A spherical particle with r₁≥15λ with a permittivity value (e.g., dielectric particle without gain) above the highest eigenpermittivity. Situating such particles that can attach to cancer cells in a sample/body and then using an external field to excite them. Alternatively, particles that are tuned to a frequency that corresponds to a frequency in which target cells/target cell-components are active/resonant possibly with an external field. A spherical particle as a detector—when a source is active at given direction it will direct its radiation towards this direction, enabling to point to its direction. Then, it is possible also to direct additional radiation to this direction to eliminate the source of radiation (e.g., as a defensive weapon).
6. Combination of a large spherical layer and a large spherical particle both with a multi-resonance permittivity for imaging and ablation purposes.
7. A structure composed of a combination of concentric spherical structures such as two or more spherical layers, spherical layer(s) and a sphere, that can respond to many modes simultaneously.

### INDUSTRIAL APPLICABILITY

1. The spherical layer setup can be used in imaging and ablation. For imaging purposes the signal can be collected outside of the spherical layer by a means of a lens or a detector. The source for this purposes can an external source such as a laser situated outside of the spherical layer or polarization of a medium at the origin. Large spherical layers can be capped to remove the degeneracy and generate only the first TM mode. This can be used by pharma companies in drug development and optimization, imaging biological samples. In addition, it can be used to ablate cancer cells by using infrared frequencies that can penetrate more into the human body and ablate materials to generate three-dimensional shapes.
2. A spherical particle in a three-body resonance mechanism can be without gain. It can be used both for imaging such particles or ablating near such particles (at the origin).
3. The enhancement of light-matter interactions when an atom or molecule, are situated at the origin can be used to image a certain type of atoms or molecule at a certain frequency. The setup can be tuned to the frequency with the Rabi splitting so that it will be specific for this molecule at the origin. This can also be used to image biological samples and heat the surrounding of the atoms and molecules (and possibly ablate them).
4. The setups above can also be used to image or ablate in larger systems such as the human body.
5. The spherical particles with the high degeneracy of the eigenpermittivities can be used to target cancer cells in their environment or interact specifically with relevant cell components through a specific frequency (e.g., cancer cells or cell components of cancer cells that are more susceptible to certain frequencies). This can be done by exciting the spherical particles with an external source or by enhancing emission and absorption of nearby atoms and molecules. They can also be used for ablation of cells when they are at the origin using the spherical layer and possibly utilizing a system resonance to enable field localization. In addition, such particles can function as omnidirectional antenna/detectors for various applications such as radars, three dimensions detectors in security etc.

### DETAILED DESCRIPTION

Eigenfunctions of Maxwell's equations are fields, which exist without a source for certain physical parameters that correspond to resonances of the system [26, 27]. Here, we utilize resonances in a setup of a spherical layer in a host medium to naturally generate a the VSHs. This setup requires only a point source in order to generate these field patterns. The permittivity value of the spherical layer e₁ will be close to a resonant TM l=1 permittivity value in order to generate this VSH (a resonant permittivity enables the existence of a field without a source as in a gain medium in laser). Similarly, all the other modes can be excited for the permittivity values close the eigenpermittivities, generating the time reversal of all the multipole radiation patterns, which correspond to all the emission and absorption transitions of atoms and molecules [28]. Alternatively, a frequency which is close to an eigenfrequency can be used. Using an eigenpermittivity is advantageous in this context since the resonance can be fully reached by introducing a gain. While these eigenvalues are usually associated with a gain that is needed to generate the field, there are some cases when they are real valued [29] or have epsilon near zero [29, 30].

The electromagnetic field expansion for a physical electric field E at a given angular frequency ω can be written as follows [26]

$$E = {E}_{0} + {\sum }_{n} \frac{{s}_{n}}{s - {s}_{n}} \frac{〈 {\tilde{E}}_{n} \vert {E}_{0} 〉}{〈 {\tilde{E}}_{n} \vert {E}_{n} 〉} \vert {E}_{n} 〉 , \qquad ( 1 )$$

where sₙ≡∈₂/(∈₂-∈₁ₙ) is the eigenvalue, ∈₂ is the host-medium permittivity, s=∈₂/(∈₂-∈₁) Eₙ and \tilde{E}ₙ are the eigenfunction and its dual, and E₀ is the incoming field. [symbol] E₁|E₂ [symbol] =∫drθ₁ (r) E₁·E₂ and θ₁ (r) is a window function which equals 1 inside the inclusion volume. Thus, when ∈₁ is close to ∈₁ₙ, 1/(s-sₙ) >>1 and the corresponding eigenfunction has a large contribution in the electric field expansion (see for example Ref. [31]FIG. 2). Clearly, other modes and the incoming field exist in the expansion. Fortunately, close to a resonance, the TM l=1 eigenfunction will have the dominant contribution inside the spherical volume.

Still, VSHs have a degeneracy in the m index, which usually results in the generation of all the m modes as a response to an incoming electric field. We therefore employ the current formulation of the field expansion in order to remove this degeneracy. In this formulation we express the incoming field in terms of Green's tensor E₀(r)=∫dV [symbol] (r, r′) ·J (r′) and substitute it in [symbol] \tilde{E}ₙ|E₀ [symbol] . Then, we change the order of integration and use the definition of the eigenfunction to obtain [29]

$$〈 {\tilde{E}}_{n} \vert {E}_{0} 〉 = - \frac{4 π i}{{∈}_{2} ω} \int d {V}^{′} {θ}_{1} ( {r}^{′} ) {E}_{n} ( {r}^{′} ) \cdot \int d V \overleftrightarrow{G} ( {r}^{′} , r ) \cdot J = - \frac{4 π i}{{∈}_{2} ω} {s}_{n} \int {dVE}_{n} ( r ) \cdot {J}_{dip} ( r ) = - \frac{4 π {s}_{n}}{{∈}_{2}} p \cdot {E}_{n} ( {r}_{0} ) ,$$

where J_{dip} (r) is an oscillating point electric dipole, p is the dipole moment, and ω is the oscillation frequency.

Now the expansion of the electric field reads

$$E = {E}_{0} - \frac{4 π}{{∈}_{2}} {\sum }_{n} \frac{{s}_{n}^{2}}{s - {s}_{n}} \frac{p \cdot {\tilde{E}}_{n} ( {r}_{0} )}{〈 {\tilde{E}}_{n} \vert {E}_{n} 〉} \vert {E}_{n} 〉 . \qquad ( 2 )$$

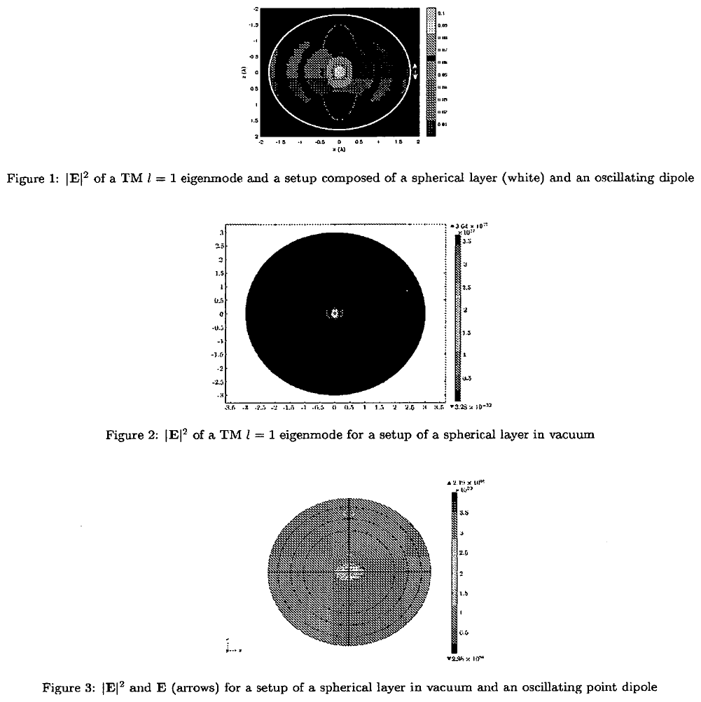

Thus, situating an oscillating dipole may result in the generation of one TM l=1 mode (see FIG. 1).

The general form of a TM VSH is [1]

$${E}_{l m}^{( n )} ∝ \frac{1}{∈ ( r )} ∇ \times {f}_{l} ( kr ) {X}_{l m} , {X}_{l m} = \frac{1}{\sqrt{l ( l + 1 )}} {LY}_{l m} , \\ {f}_{l} ( kr ) = {A}_{l}^{( 1 )} {h}_{l}^{( 1 )} ( {k}_{r} ) + {A}_{l}^{( 2 )} {h}_{l}^{( 2 )} ( kr ) ,$$

where fₗ (r) is a linear combination of spherical Hankel functions, hₗ (r) is a spherical Hankel function, k is the wavevector,

$$L = \frac{1}{\text{?}} ( r \times ∇ ) , \\ \text{?} \text{indicates text missing or illegible when filed}$$

and Yₗₘ is a spherical harmonic.

For a spherical layer in r₁<r<T<r₂, fₗ (r) that satisfies boundary conditions is of the form

$${f}_{l} ( r ) = \{ \begin{matrix}{C}_{l} {h}_{l}^{( \cdot )} ( {k}_{2} r ) & r > {r}_{2} \\ {B}_{l}^{( l )} {h}_{l}^{( 1 )} ( {k}_{1 n} r ) + {B}_{l}^{( 2 )} {h}_{l}^{( 2 )} ( {k}_{1 n} r ) & {r}_{1} < r < {r}_{2} \\ {A}_{l} {j}_{l} ( {k}_{2} r ) & r < {r}_{1}\end{matrix} ,$$

where jᵢ (r) is a spherical Bessel function and k₁ₙ, k₂ correspond to ∈₁ₙ, ∈₂, respectively. These eigenfunctions are standing waves for r<r₁ and propagating waves for r>r₂ at a given frequency. The eigenpermittivity ∈₁ₙ in r₁<r<r₂ is calculated using an eigenvalue equation as we now explain.

An eigenpermittivity enables the existence of the field without a source and we therefore only need to impose boundary conditions. From continuity of tangential E and H we have for a TM eigenfunction (assuming ∈₂=1)

$${f}_{1} ( {r}_{1}^{-} ) = {f}_{1} ( {r}_{1}^{1} ) , {f}_{1} ( {r}_{2}^{-} ) = {f}_{1} ( {r}_{2}^{1} ) , \\ \frac{\partial ( {rf}_{1} ( r ) )}{\partial r} {\vert}_{r - {r}_{1}} = \frac{1}{ϵ \text{?}} \frac{\partial ( r {f}_{1} ( r ) )}{\partial r} {\vert}_{r - {r}_{1}^{1}} ;$$

$$\frac{1}{{ϵ}_{1 n}} \frac{\partial ( {rf}_{1} ( r ) )}{\partial r} {\vert}_{r - {r}_{2}^{-}} = \frac{\partial ( {rf}_{1} ( r ) )}{\partial r} {\vert}_{r - {r}_{2}^{+}} ;$$

$$\text{?} \text{indicates text missing or illegible when filed}$$

from which we obtain an eigenvalue equation and ∈₁ₙ. Similarly for a TE eigenfunction we write

Eₗₘ^{(M)∝f}ₗ (kr) Xₜₘ;

with the boundary conditions

$${f}_{1} ( {r}_{1}^{-} ) = {f}_{1} ( {r}_{1}^{+} ) , {f}_{1} ( {r}_{2}^{-} ) = {f}_{1} ( {r}_{2}^{+} ) , \\ \frac{\partial ( r {f}_{1} ( r ) )}{\partial r} {\vert}_{r - {r}_{1}} = \frac{\partial ( r {f}_{1} ( r ) )}{\partial r} {\vert}_{r - {r}_{1}} , \\ \frac{\partial ( r {f}_{1} ( r ) )}{\partial r} {\vert}_{r - {r}_{2}^{-}} = \frac{\partial ( r {f}_{1} ( r ) )}{\partial r} {\vert}_{r - {r}_{2}^{-}} ,$$

Clearly, the eigenpermittivities of the TE and TM modes depend on the radius and the thickness of the spherical layer.

The eigenfunctions in the radiation zone (far field) can be expressed as [1]

Eₗₘ^{TM}→Z₀Hₗₘ^{TM}×n,

where n=r/r. Hence, since Hₗₘ^{TM}∝Eₗₘ^{TE} is parallel to the sphere surface [1], Eₗₘ^{TM} is also parallel to the sphere surface. Thus, due to the inner product in Eq. (2), when an oscillating dipole is placed in the radiation zone it may excite a mode if it is oriented parallel to the spherical-layer surface.

For concreteness, we situate an oscillating dipole outside the spherical layer on the positive x axis. The y, z components of Hₗₘ^{TM} can be found from [1]

$${H}_{l m}^{TM} ∝ {LY}_{l m} , {L}_{y} = \frac{1}{2 i} ( {L}_{+} - {L}_{-} ) , {L}_{z} {Y}_{l m} = {mY}_{l m} .$$

The z components of the TM l=1 eigenfunctions in the radiation zone readily follow from the two relations above

E_{l-t,m-0 z}^{TM}≠0, E_{l-1,m-±1 z}^{TM}=0.

Thus, by placing an oscillating dipole on the n axis directed along the z axis we have removed the nt degeneracy of the TM modes. It can be seen that objects at all locations will generate a focal spot at the origin. In addition, the θ dependency of the field can be written as E_{TM l-1} (θ) ∝sin θ (−\grave{x}cos θ+\grave{z}sin θ) which equals the θ dependency of the far field of an oscillating dipole and shows that the mode is indeed its time reversal. Oscillating dipoles on the xy plane directed along z will generate fields in the z axis at the focal spot. From symmetry, situating several current sources will result in a superposition of the TM l=1, m=0 mode according to their locations and orientations. In addition, other forms of illumination (which correspond to current distributions) such as a laser illumination may also be used to generate a subwavelength focal spot (the current source may be associated with the gain medium). Also, since [1]

$${\sum }_{\text{?} - l}^{l} {\vert {X}_{l m} ( θ , φ ) \vert}^{2} = \frac{2 l + 1}{4 π}$$

$$\text{?} \text{indicates text missing or illegible when filed}$$

combining two spherical structures (e.g., a sphere and a spherical layer), each corresponding to a TM l=1 resonance at a given frequency, and using oscillating dipoles such that all the m modes are excited, will result in isotropic radiation.

In order to have a dominant contribution of the TM l=1 modes, the physical permittivity has to be much closer to the corresponding eigenpermittivity compared with its distances from the eigenpermittivities of the other modes. The high-order modes have a minor contribution to the expansion and we can focus on a certain l range when comparing these distances [32]. The resonant permittivity usually has an imaginary part that corresponds to gain. While incorporating gain in the spherical layer will bring the system to a resonance, if a real-valued permittivity will be close enough to a resonance, a similar effect is expected. The spacing between resonances and the imaginary part of the permittivity depend on the thickness of the spherical layer. A thin spherical layer will result in a large eigenpermittivity gain and widely-spaced resonances. A thick spherical layer will result in a small imaginary part of the eigenpermittivities and more closely spaced resonances.

When the system is close to a resonance and there is a polarizable/absorbing medium at the origin, the dominant contribution to the electric field can be from the emission at the origin. From Eq. (2) it can be seen that the source location near the origin translates into Ēₙ (r₀) and the source magnitude is proportional to Eₙ(r₀). We thus get that when the field is generated by the medium at the origin there is an additional factor of \sqrt{2} in the effective FWHM in each dimension.

An additional degeneracy arises when r₁, r₂≥10λ since at the r>>λ limit jᵢ, hₗ⁽¹⁾ have the form

$${j}_{l} ( r ) \to , \frac{1}{r} sin ( r - \frac{l π}{2} ) , {h}_{l}^{( 1 )} ( r ) \to {( - i )}^{l + 1} \frac{{ϵ}^{ir}}{r} .$$

As a result the even and odd eigenvalues will be almost identical. A possible way to remove this degeneracy is to slightly change the structure so that the eigenfunctions and the eigenvalues will change. For example, the spherical layer can be capped from above (or in several places), which will also enable to easily place objects inside. Alternatively, this high degeneracy can be utilized for a strong optical response of the system (e.g., strong scattering, enhancement of spontaneous emission etc.). This degeneracy is an asymptotic. degeneracy and is in addition to the m degeneracy so it includes a very large number of modes. In practice, an excitation at a given frequency can excite all the even/odd TE/TM modes. Similarly, such a degeneracy is also expected for a sphere inclusion and possibly cylindrical structures. Combining spherical structures may result in an all-mode degeneracy and further enhance the response of the system. Note that the total radiated power is a sum of the contributions of all the multipoles [1].

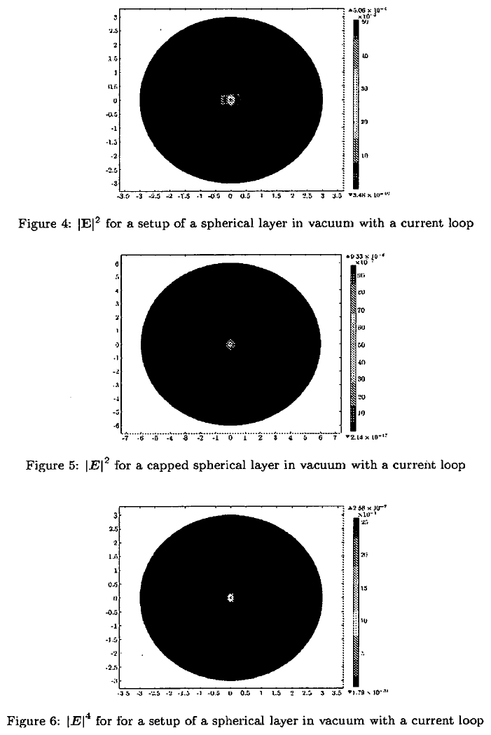

To cross validate our analysis we calculated for setups of spherical layers in vacuum the eigenmodes and |E|² as a response to an excitation of a dipole and a current loop using Comsol. In FIG. 2 we present a TM l=1 mode for r₁=0.7, r₂=1.4 μm, ∈₁=1.5, ∈₂=1, and ω_{TM l-1}/2π=7.92781·10¹⁴+7.397·10⁷i, where ω_{TM l-1} is an eigenfrequency. It can be seen that the focal-spot size (normalized by λ) matches the one in the analytical calculation presented in FIG. 1. Eigenmodes exist without a source, which in the eigenpermittivity formulation arises from gain in the spherical layer, similarly to a laser. In addition, ωₗ₋₁ is almost real and we therefore expect that at ω=Re(ωₗ₋₁), ∈₁ₗ≈1.5 will be almost real. The eigenfrequencies in this case are closely spaced, which requires high precision in e₁ to obtain a resonance. We then considered a setup of r₁=0.7, r₂=0.9 μm, ∈₂=1, λ=430 nm and an oscillating point dipole parallel to the spherical layer. We calculated ∈_{TM l-1} using the TM eigenvalue equation around ∈ₗ₁=1.5 and substituted the result rounded to two digits after the decimal point as the physical permittivity ∈₁ in a Comsol simulation. In FIG. 3 we present |E|² and E (arrows) for ∈₁=1.75-0.7i, r₀=1\hat{x}μm, p=1\hat{z}mA in axial cross section. It can be seen that the focal-spot normalized size matches the ones in FIGS. 1 and 2. Situating the dipole at any other distance will also result a focal spot at the origin, unlike imaging using a lens. In FIG. 4 we present |E|² for a setup with a current loop with ∈₁=1.45-0.57i, ∈₂=1, r₁=2 μm, λ=430 nm, J=1\hat{z}zA/m², and r₀=2.2 μm. Interestingly, the field intensity is much stronger at the origin compared to the one around the current loop. In addition, the current distribution reminds a gain medium distribution in a laser, which may mean that a laser can also be used to generate this TM l=1 mode. In FIG. 5 we demonstrate focusing using a capped spherical layer with r₂≈10λ. This structure has a full azimuthal-angle coverage unlike focusing light using two lenses. In all the simulations we used a perfectly matched layer to account for boundary conditions (external layer). The focal spot size is similar to the one in the complete spherical layer (smaller in volume by a factor of 18 compared to a focal spot that can be generated by uniformly illuminating a lens). In FIG. 6 we present |E|^{λ} for for a setup of a spherical layer in vacuum with a current loop and ∈₁=1.45-0.57i, r₁=1.7, r₂=2 μm, λ=430 μm, r₀=2.2 μm. |E|⁴ represents the effective intensity in excitation-collection mode as explained in the manuscript. As can be seen the effective focal spot is smaller by a factor of \sqrt{2} in each dimension and the side lobes are negligible.

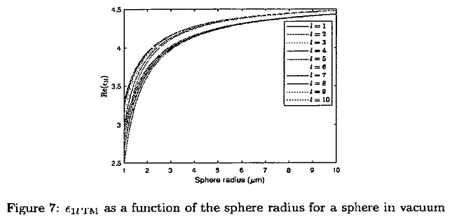

Now we analyze the TM eigenpermittivities for a sphere inclusion in vacuum as we increase the sphere radius. Similarly to the spherical-layer setup all the odd/even eigenvalue equations coalesce when increasing the sphere radius r₁. In FIG. 7 we present ∈_{l1TM} as a function of r₁ for λ=430 nm. The eigenpermittivities have a negligible imaginary part (smaller than 10⁻⁸) and we therefore present only the real part. It can he seen that for r₁>8 μm all the even/odd eigenvalues are practically the same. Thus, using a physical permittivity ∈₁ that is close to the odd or even eigenpermittivity, will excite all (or most) of these eigenstates, leading to a very strong response of the system (without requiring gain in this case). Note that at large sphere radii the eigenvalues are more robust to changes in the radius.

We now evaluate the enhancement of various optical phenomena due to the infinite-asymptotic degeneracy. We investigate the enhancement of spontaneous emission [33] of a dipole in a sphere/spherical-layer setup when r₁, r₂, r_{dipol}>>λ due to the infinite degeneracy. To that end, we write the expression for the density of states [34-36], which is dominant in Fermi-Golden-Rule calculation [37]

$${ρ}_{p} = \frac{2 ω}{π} Im [ G \text{?} ( r , {r}^{′} , ω ) ] . \\ \text{?} \text{indicates text missing or illegible when filed}$$

We then evaluate the sum in the eigenfunction expansion in Eq. (2). We consider a sphere with a physical permittivity that is slightly above the first or second eigenpermittivity, namely ∈₁>∈₁₁ or ∈₁>∈₁₂ (see FIG. 5). In this situation sₗ²/(s-sₗ) have the same sign and approximately the same value for a very large number of modes (e.g., at least 20 modes for r₁, r₂≥40λ). We now analyze Ê_{l,n} (r_{dipole})Eₗₚ(r_{dipole}). Since the dual eigenfunctions [26]

E̅ₗₘ^{(E)}∝∇×f₁ (kr) Xₗₘ*, E̅ₗₘ^{(M)} ∝fₗ (kr) Xₗₘ*;

we get that the phase of Ē_{lμ} (r_{dipole})E_{lμ}(r_{dipole}) is determined by fₗ. Since fₗ≈fₗ₊₂ we get approximately the same phase for all the modes whose eigenvalues coalesce. Similar arguments apply for the inner product [symbol] \tilde{E}ₙ |Eₙ [symbol] , see Appendix A in Rsf. [26]. For example, the integral in the inner product of the TE modes can be performed analytically and can be shown to be invariant to l→l+2. This leads to a constructive interference in the field summation. Thus, if we have modes that have effectively the same eigenvalue, their resonance constributions will add constructively and we get approximately n times enhancement in the density of states compared with a standard resonance of the same structure. Clearly, the larger the spherical-structure radius, the more eigenvalues will be effectively the same (see FIG. 7). This should be multiplied by the enhancement factor that arises from the proximity of the physical permittivity to the resonant permittivity (∝1/(s-sₙ) from Eq. (2)). See Ref. [31] in which the modes also interfere constructively. In this reference when the physical permittivity is close to the first eigenpermittivity sₗ²/(s-sₗ) decays upon increasing l and when the physical permittivity is close to the accumulation point the field is enhanced very close to the metal-dielectric interface since the high-order modes, which have approximately the same sₗ²/(s-sₗ), decay spatially rapidly. Here the modes have approximately the same sₗ²/(s-sₗ) contribution and they all scale as 1/r at large distances, leading to a strong response that extends relatively far from the dielectric sphere.

We proceed to analyze the enhancement of absorption and stimulated emission induced by a dipole on itself due to the infinite degeneracy. Clearly, the enhancement of the density of states in Fermi-Golden-rule calculation [37] will be the same. Assuming that the multiple expansion for light-matter interaction holds and the dipole interaction is the dominant interaction for all the modes in the field expansion, we get that if n modes are effectively on resonance and the field that is generated by the dipole is enhanced by a factor of n, |( [symbol] ψⱼ|Hᵢₙₗψᵢ [symbol] |n² will scale as rat and the overall enhancement will scale as n³ compared with a standard resonance. This is a very large enhancement and for n=20 we get an enhancement factor of 8000 (needs to be multiplied by ∝1/(s-sₙ)³). Note that a sphere with r₁=20λ is of the order of a human cell for visible and infrared light and hence this phenomenon has potential use in biomedical applications such as targeting cells with light (via spherical particles). Another potential application is omnidirectional antenna/detector, which directs its field pattern according to the source location.

The enhancement of the scattering arises from the fact that the total radiated power is a sum of the contributions of all the multipoles [1]. Thus, we deduce that the total power is enhanced by a factor of n, compared with a standard resonance, where n is the number of modes that are effectively on resonance. This should be multiplied by the enhancement factor that arises from the proximity of the physical permittivity to the resonant permittivity ∝1/(s-sₙ)². Similar analysis follows for the enhancement of absorption by a sphere as the absorption power is given by ω·Im(∈₁)|E|²/2 [1] and many modes can he excited inside the sphere.

We now investigate two directions to localize electric field with deep subwavelength resolution. We first present a three-body-resonance mechanism in which we slightly change the permittivity value of the spherical layer to move the system away from resonance and introduce a spherical particle that will bring the system back to resonance when located at the origin. We consider a spherical layer in a host medium that is off-resonance and close to a resonance, possibly having a dielectric material with gain. We then introduce a spherical particle that when situated at the origin results in a TM l=1 resonance of the three-body system for a given permittivity value of the particle, possibly a dielectric material that is different from the host-medium permittivity, and we set the physical permittivity value of the particle to be equal to this eigenpermittivity. For a different location of the particle the system will be on resonance for a different permittivity value of the particle. This setup translates location changes of the particle to changes in the eigenpermittivity, utilizing the 1/(s-sₙ) factor to localization of the particle. We thus may achieve strong localization capability of the system—for a slight change in the location of the particle the field intensity everywhere will change significantly. To translate this idea into practical' applications one can use frequencies for which the host medium that can have in general spatially-varying permittivity, is relatively uniform/transparent.

We also suggest that the time reversal of the field emitted in a transition at the origin of an atom or molecule will spatially match the quantum transition current. It was recently suggested based on a classical wave equation analysis that when the time reversal of the field emitted by a point source impinges on a perfect absorber at the origin, the field pattern will have a 1/r scaling near the origin [12]. Now we turn to the quantum analysis. We first note that in the semi-classical quantum treatment in Ref. [28] there is a 1/r scaling in the transition-rate calculation. One can think that the time reversal of an emission process is absorption, having a field with a 1/r dependency near the origin. In practice, emission and absorption are related to the transition between electronic or nuclear eigenstates. We can thus expect that the field will not diverge and think of a classical analogue of a dipole with a characteristic size of the average distance of the probability density function from the center of mass. Let us analyze the emission process and its time reversal. We consider a hydrogen atom for simplicity and assume that there is a transition from an eigenstate ω₁ to an eigenstate ψ₂. As a result of the spatial change in the probability density function electric field is emitted. We express the quantum current

$$j = v ρ = v {\vert ψ \vert}^{2} = \frac{1}{2 m} ( {ψ}^{*} p ψ - ψ p {ψ}^{*} ) ,$$

where ψ is the wavefunction that can transition between states and v is the group velocity of the particle [37]. The electric field E then propagates in space occupying a spherical shell. Now we time reverse the process. We assume that the field is generated on the spherical shell. The field then propagates toward the atom/molecule. We assume that when the field reaches the atom or molecule they are in the same state as when they emitted the field up to a π phase difference in v. Using reciprocity and treating the quantum current as classical the field near the origin will then be in the same form of the quantum current j that generated the field. Thus, the field pattern matches the form of the transition current and can be optimal for driving the transition. The field is thus deep subwavelength with typical size of the average distance of the density function from the center of mass. In this situation the spatial variations of the electric field are comparable to the electron/nuclear wavefunction and the spatial variations of E or A will have to be taken into account explicitly in light-atom/molecule interaction calculations. In standard light-atom/molecule interaction the term

$$- \frac{q}{m} P .$$

A for the value of A at the atom/molecule location drives the dipole transition. However, A is constant [37] and not necessarily spatially overlaps optimally with the current that drives the transition. This absorption process can be complemented by stimulated emission for a field that oscillates at a frequency ω. Note that the resonant spherical layer should be tuned to this ω. This process can have unique characteristics such as strong absorption and emission, high-order multipole transitions involved, large Rabi shift/splitting etc. See for example Refs. [38-41] in which phase matching of the electric field to the electron wave function results in a stronger interaction. It would then make sense that a slight change in the position of the atom/molecule from the origin will bring the interaction to the standard multipole-expansion interaction. Hence, if this can be realized experimentally using a resonant spherical layer or another setup that can generate the time reversal of the emitted field (for example a reflecting or phase-conjugating cavity) it may enable to localize atoms/molecules with deep-subwavelength resolution (for scattering medium with ballistic photons). For example, there can he strong absorption and emission or a large Rabi splitting when the atom/molecule are situated at the origin. One can then probe these properties by e.g., observe a frequency shift possibly via the three-body resonance mechanism described above. In addition, we note that this description is applicable to all the transition types (dipole, quadrupole etc.). While it is true that the spontaneous-emission rate of high-order radiation multipoles is usually slow, when it will occur for an atom/molecule at the origin the incoming time-reversed field can spatially match the transition current and drive the transition. This can be utilized to observe a quadrupole transition that cannot be observed otherwise. Such a transition can have a different frequency to which the medium is usually transparent. This can be another mechanism to localize atom/molecules with deep subwavelength resolution. Alternatively, transitions can be driven by an external current source. In order for the spherical layer to respond to several transition orders one can utilize the infinite-asymptotic degeneracy. Note that when radiation is emitted by an atom/molecule at the origin, the spherical-layer setup generates the time-reversed field also according to the orientation, which maximizes the spatial overlap when interacting with the atom/molecule.

In addition, close to a resonance the density of states given

$${ρ}_{p} = - \frac{2 ω}{π} Im [ G \text{?} ( r , {r}^{′} ω ) ] , \\ \text{?} \text{indicates text missing or illegible when filed} \qquad [ 34 \text{-} 36 ]$$

where G_{μμ}(r, r′, ω) can be expressed as the electric field due to a dipole at the dipole location and direction in Eq. (2). Thus, when approaching a resonance the density of states and the field increase and as a result the transitions are enhanced. Hence, quantum mechanically we have enhancement in two aspects: field overlap with the transition current and increase in the density of states and electric field.

We introduced a setup of a spherical layer, that close to a resonance generates the time reversal of the atomic and molecular multipole transitions. The time reversed signal in our setup is spatially continuous and is naturally generated by a medium with a uniform permittivity.

We started by situating the spherical layer in a uniform medium, which generates a subwavelength free-space focal spot in three dimensions. The degeneracy of the excited mode is removed by incorporating currents on a plane which is perpendicular to the spherical layer. Such currents can he realized by a medium, which is polarized due to an impinging electric field or even a laser source. Interestingly, when situating an object at the origin the field emitted by the polarized medium at the focal spot excites the TM l=1 spherical layer mode, which reexcites the medium at the focal spot etc. This coupling can enhance the emission from the medium at the focal spot. Also, near a resonance the field becomes very strong and may enable larger penetration of ballistic photons and enhancement of the signal generated at the focal spot by the spherical layer. To image from the focal spot, one can think of collecting light from the other side of the spherical layer by means of a lens or another optical element. This signal is mostly composed of the sum of the excitation of the TM l=1 mode due to the sources and the polarized medium at the focal spot, which may enable to acquire also the phase in the measurement. To further minimize the effective focal-spot size techniques such as nonlinear optics, PALM or STORM [7, 8], and quantum imaging [42] can be used. In addition, the TM l=2 and TE l=1 modes have a torus shape [25] and may be used to stimulate fluorescence emission at another wavelength similarly to STED [6].

We then suggested two directions to localize field with deep subwavelength resolution. We presented a three-body-resonance mechanism in which we slightly change the permittivity value of the spherical layer to move the system away from resonance and introduce a spherical particle that will bring the system back to resonance when located at the origin. We then situated an atom or molecule at the origin and considered the possibility that the time reversed field of a transition will generate field near the origin that spatially correlates with the quantum-transition current, resulting in a much stronger interaction at the origin.

The resonant spherical shell setup differs from a spherical cavity in several aspects: 1. It enables light from outside of the spherical shell to generate field inside and vice versa. 2. There is a strong amplification of the signal. Thus, even spontaneous emission can generate substantial field at the focal spot. When the system is on resonance, the mode is generated without a source. 3. It couples to a single multipole or equivalently an atomic/molecular transition spatially and temporally.

This analysis is applicable to all wavelengths and due to its wave nature it may also apply to acoustics, in which gain materials were recently introduced [43], and matter waves. In addition, each spherical-layer mode has several eigenvalues and therefore there is fie3dbility in choosing the spherical-layer material, which may have importance for frequencies where it is more challenging to find materials that can focus waves [44]. Importantly, it was shown that spherical waves (VSHs) can be generated by a single source, which may enable their practical generation, also at high frequencies where current modulation is impractical. Potential applications are high-resolution 3D imaging and precise tissue ablation. In addition, the fact that this setup has a very high Q factor may be utilized to cavity QED, entanglement, and quantum information [45]. Finally, for spherical structures with r₁≥10λ there are all-odd and all-even TM/TE eigenvalue degeneracies, which results in a variety of optical phenomena of the system close to one of these eigenvalues. Combining spherical structures e.g., a sphere and spherical layer(s), each with a permittivity close to one of these resonances, may even result in an all-mode resonance.
[1] J. D. Jackson. Classical electrodynamics. John Wiley & Sons, 2012.
[2] M. Minsky, Scanning 10 1988
[3] C. B Muller and J. Enderlein PRL 104 19 2010
[4] J. Rho, Z. Ye, Y. Xiong, X. Yiu, Z. Liu, H. Choi, G. Bartal, and X. Zhang Nature communications 1 143, 2010
[5] Christoph Cremer and Thomas Cremer. Microscupica acts, pages 31-44, 1974.
[6] S. W. Hell and J. Wichmann, Optics letters, 19(11):780-782, 1994; V. A. Okhonin. Patent SU, 1374992, 1986.
[7] E. Betzig, G. H. Patterson, R. Sougrat, O. W. Lindwasser, S. Olenych, J. S. Bonifacino, M. W. Davidson, J. Lippincott-Schwartz, and H. F. Hess, Science, 313(5793):1642-1645, 2006.
[8] W. E. Moerner and L. Kador. PRL, 62(21):2535, 1989.
[9] O. Bitton, R. Bruch, and U Leonhardt, Physical Review Applied 10 4 044059, 2018
[10] M. A Alonso, New Journal of Physics 17 7 073013, 2015
[11] G. Lerosey, J. De Rosny, A. Tourin, M. Fink Science 315 5815 (2007).
[12] G. Ma, X. Fan, F. Ma, J. de Rosny, P. Sheng, and M. Fink, Nature Physics 14 6 608, 2018
[13] J. B. Pendry, Physical review letters 85 18 3966 2000
[14] M. Fink, IEEE transactions on ultrasonics, ferroelectrics, and frequency control 39 5, 555-566, 1992.
[15] G. Lerosey, J De Rosny, A Tourin, A Derode, G Montaldo, and M Fink, PRL 92 19 193904, 2004
[16] H. Noh, Y. Chong, A. D. Stone, and H. Cao, PRL 108, 186805 (2012)
[17] L. D. Landau and E. M. Lifshitz, Quantum Mechanics: Non-relativistic theory, volume 3.
[18] C. M Bender and S. Boettcher, PRL 80 24, 1998.
[19] R. El-Ga,nainy, K. G. Makris, D. N. Christodoulides, and Z. H. Musslimani, Optics letters 32 17 2632-2634 2007
[20] C. E. Ruter, K. G. Makris, R. El-Ganainy, D. N Christodoulides, M. Segev, and D. Kip, Nature physics 6 3 192 2010
[21] Z. Lin, A. Pick, M. Lontar, and A. W Rodriguez, PRL 117 10, 2016
[22] W. R. Sweeney, C. W. Hsu, S. Rotter, and A. D. Stone, Phys. Rev. Lett. 122 093901, 2019
[23] D.J. Bergman J. Phys. C: Solid State Phys. 12, 1979.
[24] A. Pick, B. Zhen, O. D. Miller, C. Hsu, F. Hernandez, A. W. Rodriguez, M. Soljacic, and S. Johnson, Optics express, 25 11, 2017.
[25] A. Farhi and D. J. Bergman, Physical Review A, 96(2):023857, 2017.
[26] D. J. Bergman and D. Stroud, Physical Review B, 22(8):3527, 1980.
[27] L. Ge, Y. D. Chong, and A. D. Stone, Physical Review A 82 6 063824, 2010
[28] E. U. Condon, The Astrophysical Journal 79 217, 1934
[29] A. Farhi and D. J. Bergman, Physical Review A, 93(6):063844, 2016.
[30] A. Alu, M. G. Silveirinha, A. Salandrino, and N. Engheta, Physical Review B 75, 15 155410, 2007.
[31] A. Farhi and D..1 Bergman, Physical Review A 96 4 043806, 2017.
[32] C. F. Bohren and D. R. Huffman, Absorption and scattering of light by small particles. John Wiley & Sons, 2008.
[33] M. S. Egglestona, K. Messera, L. Zhangb, E. Yablonovitcha, and M. C. Wua PNAS 115 (2005).
[34] A. Taflove, A. Oskooi, and S. G. Johnson, Advances in FDTD Computational Electrodynamics: Photonics and Nanotechnology (Artech House, 2013), arXiv:1301.5366.
[35] F. Wijnands, J. B. Pendry, F. .T. Garcia-Vidal, P. M. Bell, L. M. N. Moreno, and P. J. Roberts, Opt. Quant. Elect. 29, 199-216 (1997)
[36] A. Lagendijk and B. A. V. Tiggelen, Phys. Rep. 270, 143-215 (1996)
[37] C. Cohen-Tannoudji, B. Diu, and F. Laloe. Quantum Mechanics. Wiley (1977)
[38] P. Agostini et al., Phys. Rev. Lett. 42, 1127 (1979),
[39] P. B. Corkurn and F. Krausz, Nat. Phys. 3, 381-387 (2007).
[40] S. Ghimire and D. A. Reis, Kat. Phys. 15, 10-16 (2019).
[41] R. Dalian, S. Nehemia, M. Shentcis, O. Reinhardt, Y. Adiv, K. Wang, O. Beer, Y. Kurman, X. Shi; M. H. Lynch, and I. Kaminer, arXiv:1909.00757 (2019).
[42] R. Tenne, U. Rossman, 13. Raphael, Y. Israel, A. Krupinski-Ptaszek, IL Lapkiewics, Y. Silberberg, and D. Oron, Nat. Phot., 41566 018 0324, 2018.
[43] W. Morten and C. Johan, Physical Review B, 89 4 041201, 2014.
[44] O. Graydon, Nature Photonics 12 5 254, 2018
[45] J. M. Raimond, M Brune, and S. Haroche, Reviews of Modern Physics 73 3, 2001.

1. An apparatus comprising of a spherical shell in a host medium, wherein the host medium can be comprised of several components such as air and a sample, and wherein a focal spot is generated.

2. The apparatus according to claim 1, used for imaging and ablation, wherein for imaging electric field is collected outside of the spherical shell.

3. The apparatus according to claim 1, wherein the physical permittivity of said spherical shell is uniform and close to an eigenpermittivity.

4. The apparatus according to claim 1, wherein said spherical shell is capped in one or more places.

5. The apparatus according to claim 1, wherein a particle that results in a system resonance when it is situated at the origin is introduced, thereby generating large field intensity when it is at the origin.

6. The apparatus according to claim 5, wherein said apparatus without said particle is off-resonance.

7. The apparatus according to claim 1, wherein an external source excites said spherical shell, which generates said focal spot.

8. The apparatus according to claim 1, wherein when an atom or a molecule is situated at the origin they experience strong light-matter interaction.

9. The apparatus according to claim 8, wherein said apparatus with said atom or molecule at the origin is tuned to be on resonance.

10. The apparatus according to claim 8, wherein said apparatus without said atom or molecule at the origin is off-resonance.

11. The apparatus according to claim 1, wherein said spherical shell has a radius larger than the considered electromagnetic wavelength and a physical permittivity value dose to multiple eigenpermittivities.

12. A device of a sphere with a radius larger than the considered electromagnetic wavelength and a physical permittivity close to multiple eigenpermittivities, wherein multiple modes of said sphere are excited by an external source.

13. The device according to claim 12, wherein the radiation emitted by said sphere is directed towards an external source, and wherein said sphere can be used as an omni-directional detector or antenna.

14. The device according to claim 12, wherein said sphere is used to ablate human cells or human-cell components.

15. The apparatus according to claim 11 with the device according to claim 12, wherein said apparatus with said sphere are on-resonance when said sphere is at the origin.

16. The apparatus and device according to claim 15, wherein said apparatus without said sphere device is off-resonance.

17. The apparatus and device according to claim 15, wherein said sphere is used to ablate human cells or human-cell components.

18. The apparatus according to claim 3 used for optical tweezing.

19. The apparatus according to claim 3 used for quantum operations.

20. A spherical device containing multiple concentric spherical structures, possibly with radii larger than the considered electromagnetic wavelength, that can be excited with many modes simultaneously.
